(19) United States
(12) Patent Application Publication
(10) Pub. No.: US 2007/0190590 A1
(43) Pub. Date: Aug. 16, 2007

(54) Information acquisition apparatus on concentration of thioredoxins in sample, stress level information acquisition apparatus and stress level judging method

(75) Inventors: Wataru Kubo, Tokyo (JP); Tsuyoshi Nomoto, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/700,853

(22) Filed: Feb. 1, 2007

(30) Foreign Application Priority Data
Feb. 10, 2006 (JP) 2006-034350

(51) Int. Cl. C12Q 1/26; G06G 7/48

(52) U.S. Cl. 435/25; 703/11

(57) ABSTRACT

The invention is to provide an information acquisition apparatus for acquiring information relating to at least one of an oxidized form concentration, a reduced form concentration and a ratio of the concentrations of thioredoxin, useful for judging a stress level, and a stress level information acquisition apparatus and a stress level judging method utilizing the same. A reaction by an enzyme or the like catalyzing an redox reaction of thioredoxins is used to measure at least one of an oxidized form concentration, a reduced form concentration and a concentration ratio of thioredoxins, and data of such measurement is used for judging the stress level of a subject person.

### BACKGROUND OF THE INVENTION

1. Field of the Invention

The present invention relates to an information acquisition apparatus for acquiring information on a concentration of thioredoxins, and a stress level information acquisition apparatus and a stress level judging method utilizing the same. More specifically, it relates to a measuring method for measuring concentrations of oxidized form and reduced form of thioredoxin in distinguished manner, and an apparatus and an application thereof for acquiring information on concentration of thioredoxins utilizing such measuring method.

2. Description of the Related Art

An individual organism or a cell is subjected to stresses of various types. Also mechanisms of response to such stresses have a mutually common element, and many signal paths are simultaneously activated and execute regulations under close cooperation. Among such stresses, an oxidative stress represents an important index. Causes for the oxidative stress include reactive oxygen species, ultraviolet light, radiation, chemical substances and stimulations generating reactive oxygen species in a cell. The oxidative stress is known to induce a modification or a lesion to a lipid, a protein, DNA or the like in the organism. Such influence, with a strong level, results in a functional disorder or a death of the cell, and may lead to a cancer, an aging, sclerosis, dementia, or neural diseases. On the other hand, mechanisms for repairing such lesions are also known to be present. In an organism subjected to an oxidative stress, an anti-oxidative mechanism or an anti-oxidative substance is induced to repair the damaged substance in the molecular level, thereby protecting the organism. Among such anti-oxidative substances, thioredoxin is known as one of representative proteins sustaining the anti-oxidative mechanism in the cell.

Thioredoxin was discovered in 1964 as a coenzyme of rebonucleotide reductase. This enzyme is a protein having a molecular weight of 12 kDa, and has, in an active site, a SEQ ID NO:1 (Xaa: arbitrary amino acid) sequence well preserved among different species. Thioredoxin is derived by causes inducing the oxidative stress, and the concentration of thioredoxin reflects the level of an inflammation by the oxidative stress in the organism.

For measuring thioredoxin, there is reported an enzyme-linked immunosorbent assay (ELISA) utilizing an antigen-antibody reaction. For example, Non-patent Reference 1 reports that the thioredoxin concentration in serum obtained by the ELISA method is a useful index for an oxidative stress in a subject person infected by hepatitis C viruses.

Non-patent reference 1: Yoshio Sumida, Toshiaki Nakamura, Takaharu Yoh, Yoshiki Nakajima, Hiroki Ishikawa, Hironori Mitsuyoshi, Yoshikuni Sakamoto, Takeshi Okanoue, Kei Kashima, Hajime Nakamura, Junji Yodoi, Journal of Hepatology, 2000, 33, 616-622.

### SUMMARY OF THE INVENTION

As disclosed in the Non-patent Reference 1, the prior measurement for thioredoxin concentration utilizes the ELISA method. In this method, thioredoxin as a target is recognized and captured by an antibody, and the amount of captured thioredoxin is detected for example utilizing a marker. In an investigation undertaken by the present inventors on the ELISA method, it is found to involve significant fluctuations in the judgment of stress level, and requires a further improvement as a stress sensor.

As a result of further investigations on the cause of such fluctuations, it is found for the first time, for judging the stress level, that the stress level of the subject person can be more precisely judged by measuring the concentration of thioredoxins by distinguishing whether they are oxidized form or reduced form. The ELISA method measures the thioredoxin in a solution without distinguishing whether it is an oxidized form or a reduced form. It is therefore basically impossible, in the prior thioredoxin measuring methods, to measure the concentration of oxidized form or reduced form of thioredoxin or an oxidized form/reduced form ratio.

An object of the present invention is to provide an information acquisition apparatus capable of distinguishing an oxidized form and a reduced form of thioredoxin present in a sample solution and acquiring information on a concentration thereof. Another object of the present invention is to provide, utilizing such enzyme electrode, an apparatus capable of distinguishing an oxidized form and a reduced form of thioredoxin and acquiring information relating to concentrations and a concentration ratio thereof. Still another object of the present invention is, utilizing the feature of such information acquisition apparatus capable of measuring a concentration ratio of oxidized form and reduced form of thioredoxin, to provide a stress level indicating apparatus capable of providing information for classifying the stress level.

The present invention is to provide an information acquisition apparatus for acquiring information on the concentration of thioredoxins, which includes, utilizing an redox reaction of thioredoxins in a sample, to measure at least either of a concentration of an oxidized form and a concentration of a reduced form of said thioredoxins in distinguished manner.

The present invention is also to provide a stress level information acquisition apparatus for acquiring information on a stress level in a subject person of measurement, including stress level judging means which measures, utilizing an redox reaction of thioredoxins in a sample derived from the subject person of measurement, at least either of a concentration of an oxidized form and a concentration of a reduced form of said thioredoxins in distinguished manner and judges a stress level of the subject person of measurement based on first information selected from the concentration of oxidized form and the concentration of reduced form of the thioredoxins and a ratio of such concentrations, and on second information concerning a relation between the first information and a stress level.

The present invention is also to provide a stress level judging method for judging a stress level in a subject person of measurement, including a stress level judging step of judging a stress level, based, utilizing an redox reaction of thioredoxins in a sample derived from the subject person of measurement, on at least either of a concentration of an oxidized form and a concentration of a reduced form of said thioredoxins and a preset standard.

The present invention is also to provide an enzyme electrode having a conductive member and an enzyme, wherein the enzyme catalyzes an redox reaction of thioredoxins.

The present invention is also to provide a thioredoxin concentration measuring method for measuring a concentration of thioredoxins in a sample, which includes, utilizing an redox reaction of thioredoxins in a sample, to measure at least either of a concentration of an oxidized form and a concentration of a reduced form of said thioredoxins in distinguished manner.

The present invention allows to provide an information acquisition apparatus capable, on a concentration of thioredoxins, of distinguishing whether it is the concentration of an oxidized form or that of a reduced form.

Further features of the present invention will become apparatus from the following description of exemplary embodiments with reference to the attached drawings.

### BRIEF DESCRIPTION OF THE DRAWINGS

FIG. 1 is a view showing an example of a stress evaluation method utilizing two indexes and utilizing a coordinate position on a two-axis system.

FIG. 2 is a view showing an example of a stress evaluation method utilizing plural indexes and evaluating a pattern obtained on a plural-axis system.

FIGS. 3A (plan view) and 3B (cross sectional view) are schematic views of a sensing part of a thioredoxin oxidized form concentration sensor utilizing an enzyme electrode.

FIGS. 4A and 4B are schematic views conceptually showing a dependence of a current (FIG. 4A) or an integrated charge amount (FIG. 4B) measured by the enzyme electrode on a thioredoxin concentration.

FIG. 5 is a schematic view showing a series of reactions relating to reduction of thioredoxin oxidized form, taking place on the enzyme electrode.

FIG. 6 is a schematic view conceptually showing a dependence of the thioredoxin reduced form amount, measured by the enzyme electrode, on an added thioredoxin concentration.

FIG. 7 is a conceptual view of a thioredoxin oxidized form/reduced form concentration sensor utilizing a colorimetry.

FIG. 8 is a schematic view conceptually showing a dependence, on a thioredoxin concentration, of an absorbance change in colorimetry in comparison with a reference of thioredoxin.

FIG. 9 is a schematic view showing a reduction of thioredoxin oxidized form, taking place in a reaction step;

FIG. 10 is a schematic view conceptually showing a dependence of the thioredoxin reduced form amount, measured by the colormetry, on an added thioredoxin concentration.

FIGS. 11A and 11B are conceptual views showing results of stress evaluation on a hepatitis C patient and a healthy subject person utilizing a thioredoxin oxidized form concentration (FIG. 11A) and an entire thioredoxin concentration (FIG. 11B) as an index.

FIG. 12 is a conceptual view showing a two-axis evaluation utilizing, for stress evaluation, two indexes which are an entire thioredoxin concentration and a thioredoxin oxidized form/reduced form ratio.

FIG. 13 is a block diagram showing a configuration of a stress level information acquisition apparatus.

FIG. 14 is a block diagram showing a configuration of a stress level information acquisition apparatus.

FIGS. 15A (plan view) and 15B (cross-sectional view) are schematic views of a sensing part of a thioredoxin reduced form concentration sensor utilizing an enzyme electrode.

FIGS. 16A and 16B are schematic views conceptually showing a dependence of a current (FIG. 16A) or an integrated charge amount (FIG. 16B) measured by the enzyme electrode on a thioredoxin concentration.

FIG. 17 is a schematic view showing a series of reactions relating to oxidation of thioredoxin reduced form, taking place on the enzyme electrode.

FIG. 18 is a schematic view conceptually showing a dependence of the thioredoxin amount, measured by the enzyme electrode, on an added thioredoxin concentration.

FIG. 19 is a conceptual view of a thioredoxin oxidized form/reduced form concentration sensor utilizing a NADPH modified electrode system.

FIGS. 20A (plan view) and 20B (cross-sectional view) are schematic views of a sensing part of a thioredoxin reduced form concentration sensor utilizing an modified electrode.

FIGS. 21A and 21B are schematic views conceptually showing a dependence of a current (FIG. 16A) or a charge amount (FIG. 16B) in which an NDAPH modified electrode system is used on a thioredoxin concentration.

FIG. 22 is a schematic view showing a series of reactions relating to oxidation of thioredoxin reduced form, taking place on the NDAPH modified electrode.

### DESCRIPTION OF THE EMBODIMENTS

The information acquisition apparatus of the present invention for acquiring information on the concentration of thioredoxins has a structure, utilizing an redox reaction of thioredoxins in a sample, of measuring at least either of a concentration of an oxidized form and a concentration of a reduced form of said thioredoxins in distinguished manner.

Such apparatus, in a preferred embodiment, has a structure of measuring at least one selected from an oxidized form concentration, a reduced form concentration and a ratio of such concentrations, of thioredoxins. Such apparatus includes at least a reaction space for reacting the sample and an enzyme catalyzing the redox reaction of thioredoxins and detection means which calculates one or more of the aforementioned items, based on the reaction of the sample and the enzyme in the reaction space above.

The concentration measurement of thioredoxins and the acquisition of information on the concentration of the present invention include a case of determining the concentration itself in the sample and a case of determining an absolute amount of an object (compound) of measurement in the sample of a specified amount. For example sampling a blood sample of 10 ml and investigating the absolute amount thereof fall into the category of concentration measurement in the present invention.

At first, thioredoxins will be explained in detail. Thioredoxin is present in forms of an oxidized form forming a disulfide bond and a reduced form forming a dithiol, between two cysteine residues described before. Among these, the reduced form type functions as an anti-oxidative substance, not only erases the oxidative oxygen species singly but also erases the oxidative oxygen species in an interaction with peroxiredoxin, thereby becoming an oxidized form. On the other hand, the oxidized form is converted into the reduced form by the function of thioredoxin reductase and nicotinamideadeninedinucleotidephosphate (NADPH).

The enzyme which catalyzes the redox reaction of thioredoxin distinguishes whether the thioredoxin is an oxidized form or a reduced form, and catalyzes a reaction corresponding to such distinguishing. It is therefore rendered possible to measure the oxidized form concentration, the reduced form concentration or the concentration ratio of oxidized form and reduced form, which is basically impossible to measure in the prior ELISA method.

Examples of the enzyme catalyzing the redox reaction of thioredoxin include thioredoxin oxidase, thioredoxin dehydrogenase, thioredoxin reductase and peroxiredoxin, among which thioredoxin reductase is employed preferably. Such enzyme may be employed singly, or in combination with another enzyme which does not catalyze the redox reaction of thioredoxin. It is particularly preferably to employ an enzyme, which catalyzes a reaction not involving thioredoxin, in combination with the enzyme catalyzing the redox reaction of thioredoxin. Examples thereof include a combination of thioredoxin reductase and ferredoxin NADP⁺ reductase.

Thioredoxins in this case include, in addition to thioredoxin (TRX), a group of proteins having a domain for thioredoxin, called thioredoxin super family. A molecule of such thioredoxin super family has an active site constituted of SEQ ID NO:1 and executes the redox function by the disulfide/dithiol group in such active site. The thioredoxin super family also includes a molecule having plural thioredoxin motives. Examples of such thioredoxin super family include TRX, Sptrx, PDI, ERp72 and ERdj5.

The measuring method by enzyme reaction in this case is a measuring method including an enzyme reaction in the reaction for measuring the concentration of thioredoxins. The concentration of thioredoxins is measured by at least either of an enzyme reaction in which an oxyreductase is reacted with thioredoxins and an enzyme reaction linked with the redox reaction of thioredoxins. It is possible, as described above, to link, in addition to the reaction of thioredoxins by an oxyreductase, another enzyme reaction for obtaining a change detectable by detection means with the reaction of thioredoxins by oxyreductase. The enzyme to be employed in the enzyme reaction for concentration measurement may be used in a state immobilized on a carrier.

The detection method for detecting a change, obtained in the reaction space based on the enzyme reaction, is not particularly restricted. The detection method is preferably an enzyme electrode method, an absorbance method (including colorimetry) and an emitted light detection method, and more prefrably an enzyme electrode method or a colorimetry method.

Examples of the colorimetry method include a method utilizing a change in the absorbance at 340 nm when NADPH changes to NADP⁺, and a method of utilizing a dye in addition to NADPH. Examples of such dye include 5,5′-dithiobis(2-nitrobenzoic acid) and dithiothreitol.

The environment for executing the measurement is not particularly restricted, but an environment in which thioredoxins are present in a liquid is employed preferably. Also a liquid containing water, alcohols, or an ionic liquid is employed more preferably.

The enzyme electrode is constituted by including at least an electrically conductive member and an enzyme which catalyzes the redox reaction of thioredoxins. As the enzyme, another enzyme may be employed if necessary in combination with the enzyme which catalyzes the redox reaction of thioredoxins.

The electrically conductive member is used for taking out an electrical change, generated in the enzyme reaction, to the exterior for enabling measurement, and there is utilized a member constituted of a material having a high conductivity and a sufficient electrochemical stability under the conditions of executing the enzyme reaction. Examples of the material constituting such conductive member include a metal such as Au or Pt, a conductive polymer such as polyacetylenes or polyarylenes, and a metal oxide containing In, Sn or Zn. Examples of the material constituting such conductive member further include carbon materials such as graphite, carbon black, carbon nanotube, carbon nanohorn and fullerene compounds. There may also be employed a composite material formed by two or more of these materials, or a composite material formed by providing a substrate surface with a layer of a conductive material.

Method of immobilizing the enzyme on the conductive member is not particularly restricted as long as it can immobilize the enzyme so as to obtain the desired function of the enzyme electrode, and known methods can be employed for this purpose. Also in addition to the enzyme, there may be employed a mediator promoting an electron transfer between the enzyme and the conductive member, such as a metal complex, a quinone or a heterocyclic compound.

An information acquisition apparatus utilizing the enzyme electrode method of thioredoxins can be constructed by the enzyme electrode of the aforementioned constitution. Such information acquisition apparatus can be constructed at least with following elements:

(1) a reaction space capable of accommodating a sample solution;

(2) an enzyme electrode disposed in the reaction space; and

(3) reaction detection means for measuring at least one of the aforementioned items, by detecting a reaction of the sample solution accommodated in the reaction space and the enzyme electrode as an electrical change.

Such apparatus allows to measure at least one of concentrations of oxidized form and reduced form of thioredoxins and a ratio of the concentrations of oxidized form and reduced form.

The enzyme electrode method is a measuring method in which an enzyme reaction is involved in a signal detected by an electrode reaction, and specifically may be a method in which a concentration of a substance reacting on the electrode changes by an enzyme reaction, and a method in which an electrical signal between electrodes changes by an enzyme reaction. An example of the former is a method in which the concentration on the electrode of a redox substance such as oxygen, hydrogen peroxide, a quinone, or a metal complex compound changes in relation to the enzyme reaction, and an example of the latter is a method in which an impedance between electrodes changes in relation to the enzyme reaction. Also the distinction of the former and the latter is just for convenience, and a certain measuring method may belong to both. Also in the enzyme electrode, either or both of the enzyme and the substance mediating the charge transfer between the enzyme and the electrode may be immobilized on the electrode.

In case of measurement utilizing the electrode, it is preferable to increase the surface area by roughing the electrode surface or by forming a fine structured material, and to apply a treatment for reducing a background current to the electrode surface. Examples of such treatment include a chemical modification of the electrode and a physical modification thereof. Specific examples of the former include an adsorption of a thiol compound or a silane compound, and those of the latter include covering the electrode surface with a fluorinated resin film or a dialyzing film. Also these methods may be employed singly or in a combination.

A measurement, utilizing the enzyme electrode, of an electrochemical change based on an enzyme reaction involving at least either of oxidized form and reduced form of thioredoxin allows to measure the concentration of at least either of the oxidized form and the reduced form of thioredoxin. Examples of such electrochemical change include a current, a charge amount, a potential, a voltage and an impedance, of which at least one is measured to calculate the concentration of at least either of the oxidized form and the reduced form of thioredoxin. The current to be measured may be a current resulting from a reaction of a substance on the electrode, and may be a stationary current or a transient current. Also the charge amount to be measured may be an integration of the observed current, and may be a total charge or a partial charge of a specified substance in the sample solution. Also the potential to be measured may be related with a potential of an active center of the enzyme or a potential of a compound of which an redox ration changes in relation to the enzyme reaction, and may be static or dynamic. The impedance to be measured may be an impedance between the electrodes, changing in relation to the enzyme reaction, and the impedance may be evaluated by a real component, an imaginary component or a combination thereof.

Thus, in case of detecting an electrochemical change involved in an enzyme reaction, it is possible to detect such electrochemical change by at least one of current, charge amount, potential, voltage and impedance, thereby calculating the concentration of the oxidized form or the reduced form desired.

The methods of detecting the electrochemical change include following specific examples:

(A) a method of evaluating an amount of electrons, moving in relation to an enzyme reaction for reducing the oxidized form of thioredoxins, by a current or a charge;

(B) a method of evaluating an amount of electrons, moving in relation to an enzyme reaction for reducing the oxidized form of thioredoxins, by a current or a charge on an electrode, utilizing a mediating substance; and

(C) a method of evaluating an oxidized form/reduced form ratio of a mediating substance, which is oxidized in relation to an enzyme reaction for reducing the oxidized form of thioredoxin, by a current, a charge, a potential or a voltage on an electrode.

The evaluation mentioned above means to calculate the concentration of an object substance of measurement from the current, charge, potential or voltage obtained by the measurement, according to a predetermined reference, for example a calibration line.

A source of thioredoxin to be measured is not particularly restricted, but is preferably a body liquid or a tissue of an organism. Among these, a body liquid of an animal is particularly preferably employed, and a human body liquid is employed most preferably. The body liquid, though not particularly restricted, is preferably blood, a blood component (including serum and plasma), urine and saliva.

In the apparatus in such case, a separate treatment is preferably executed before and after the measurement of thioredoxins. Examples of such treatment include a separation, a segmentation, a filtration, a rinsing, an extraction, a purification, a temperature change, a dispersion, a mixing, a precipitation, a dialysis, a distillation, a modification (including a chemical reaction), an oxygen removal, a debubbling, an ultrasonic treatment, a microwave treatment, a treatment involving a magnetic field application, an electrolysis, an electrophoresis, and a chromatography. There may be executed a treatment selected from these or two or more treatments in combination, according to the necessity. It is also preferable to automate these pre- and post-treatments and incorporate them as a part of the apparatus. It is also preferable to utilize a microspace such as a micro flow path for such pre- and post-treatments and/or for measurement.

Specific examples of the measuring method utilizing the enzyme reaction include a measuring method utilizing a reductase for thioredoxins (thioredoxin reductase). In such measuring method, the thioredoxin reductase specifically reacts with the oxidized form of thioredoxins contained in the sample, thereby reducing it to a reduced form. The redox reaction in such case can be detected by various detection methods to determine the concentration of the oxidized form, distinguished from that of the reduced form. Also a thioredoxin oxidase for thioredoxins may be used in a similar manner to determine the concentration of the reduced form, distinguished from that of the oxidized form.

It is also possible, by utilizing a thioredoxin reductase, to determine the concentration of the reduced form of thioredoxins in the sample. For example, an oxidized form concentration in the sample is measured, then an oxidizing agent capable of oxidizing thioredoxins is added to the sample to convert the reduced form present in the sample to the oxidized form, and the excessive oxidizing agent is removed for example by an enzyme reaction. Then executed is a concentration measuring method, similar to the method of measuring the oxidized form concentration in the sample. Then the reduced form concentration is determined by subtracting the oxidized form concentration prior to the addition of the oxidizing agent, from the detected value.

The stress level information acquisition apparatus and the stress level judging method of the present invention are to judge a stress level of a subject person of measurement based on following first and second information:

(1) first information obtained by measuring the concentrations of oxidized form and reduced form of thioredoxins in the sample obtained from the subject person of measurement, in distinguished manner; and

(2) second information indicating a relation between the first information and the stress level.

For the first information, there may be employed at least one of the oxidized form concentration and the reduced form concentration of thioredoxins and a ratio of these concentrations. The apparatus preferably includes stress level judging means which classifies at least one of the aforementioned items relating to the concentrations of thioredoxins based on the second information, thereby judging a stress level of the subject person of measurement.

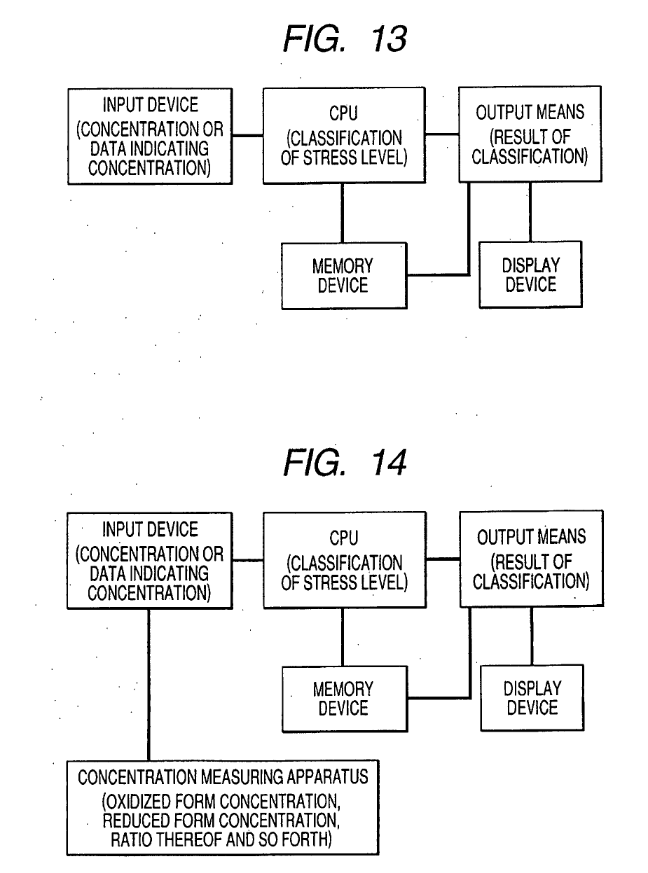

FIGS. 13 and 14 are block diagrams showing examples of constituent blocks of such apparatus.

An apparatus shown in FIG. 13 includes at least an input device, a CPU and output means, and, if necessary, further includes a memory device and a display device. A concentration value or data indicating a concentration of thioredoxins from the subject person of measurement are entered by the input device. The data indicating concentration may for example be an absorbance or a current corresponding to the detection method utilized for concentration measurement. The CPU stores in advance a program which processes the entered data according to a predetermined reference, thereby classifying and judging the stress level of the subject person of measurement. The stress level judged by such program, namely the result of judgment, may be outputted by the output means. For example, in case of employing a display as the display means, the result of judgment is displayed on the display. Otherwise, the result of judgment may be outputted on a suitable medium such as paper.

The predetermined reference for classifying the stress level may be prepared based on statistically collected data. For example, the stress level is classified into plural ranks, and a range of oxidized form concentration of thioredoxins corresponding to each rank is determined from statistically collected data. The oxidized form concentration of thioredoxins in the sample from the subject person of measurement is classified according to such ranking, and the rank of the stress level of the subject person of measurement is automatically judged by the CPU. As an index of the classification and judgment of the stress level, at least one of the oxidized form concentration and the reduced form concentration of thioredoxins and the ratio of the concentrations may be utilized. Also, if necessary, a concentration of thioredoxins (oxidized form plus reduced form) in the sample may be used additionally as an index.

A memory device provided as shown in FIG. 13 allows to store data on the thioredoxin concentration of the subject person of measurement and the result of judgment, and allows to cause the CPU to prepare data relating to a change in time, over hours, days, weeks, months or years.

An apparatus shown in FIG. 14 further includes an information acquisition apparatus for executing a concentration measurement of thioredoxins in the sample. The information acquisition apparatus preferably includes at least an apparatus executing the aforementioned measurement utilizing the enzyme reaction.

The measured value of concentration of thioredoxins, to be used in the stress level information acquisition apparatus may be obtained from followings:

(1) an oxidized form concentration of thioredoxins;

(1-1) an oxidized form concentration of thioredoxins measured utilizing a sample;

(1-2) a concentration obtained by subtracting a reduced form concentration from a total concentration of thioredoxins as a sum of oxidized form and reduced form contained in the sample;

(1-3) an oxidized form concentration obtained from a total concentration of thioredoxins as a sum of oxidized form and reduced form contained in the sample and from a concentration ratio of oxidized form and reduced form;

(2) a reduced form concentration of thioredoxins;

(2-1) a reduced form concentration of thioredoxins measured utilizing a sample;

(2-2) a concentration obtained by subtracting an oxidized form concentration from a total concentration of thioredoxins as a sum of oxidized form and reduced form contained in the sample;

(2-3) a reduced form concentration obtained from a total concentration of thioredoxins as a sum of oxidized form and reduced form contained in the sample and from a concentration ratio of oxidized form and reduced form;

(3) a concentration ratio of oxidized form and reduced form of thioredoxins;

(3-1) a ratio determined from concentrations of oxidized form and reduced form measured utilizing a sample;

(3-2) a ratio obtained from a total concentration of thioredoxins as a sum of oxidized form and reduced form and from an oxidized form concentration;

(3-3) a ratio obtained from a total concentration of thioredoxins as a sum of oxidized form and reduced form and from a reduced form concentration;

(3-4) a ratio obtained by measuring a potential of the active site of thioredoxin or by measuring a potential of a substance which changes the potential in relation to the potential of the active site.

An example of the method of determining the ratio obtained by measuring the potential of the active site of thioredoxin or by measuring the potential of a substance which changes the potential in relation to the potential of the active site is shown below.

At first, the potential measurement is executed in the presence of an electrode capable of executing an electron transfer with a sufficient velocity for measurement with the active site of thioredoxin or in the presence of a substance enabling an electrical connection between the active site and the electrode thereby enabling potential measurement of the active site of thioredoxin. Under such condition, the potential varying by the oxidized form/reduced form ratio of thioredoxin is calculated according to Nernst equation:

Nernst equation: E=E⁰+(RT/nF)ln(a₀/a_{R})

wherein:

E: electrode potential

E⁰: standard electrode potential

R: gas constant

T: absolute temperature

n: number of electrons involved in the reaction

F: Faraday constant

a₀: active amount of oxidized form

a_{R}: active amount of reduced form

Also for measuring the total amount of thioredoxins, a method not utilizing enzyme may be used in combination. Examples of such method include the enzyme-linked immunosorbent assay (ELISA).

Thioredoxin, which is an antioxidative substance, is induced when an oxidative stress is applied to the organism, thereby increasing the concentration, and is therefore used as an index of the oxidative stress as described in the Non-patent Reference 1. However, this index is an indirect index involving plural steps such as:

1. generation of oxidative stress,

2. detection of oxidative stress by the organism,

3. induction of thioredoxin, and

4. increase in the concentration of thioredoxin.

Therefore, a time difference exists from the generation of the oxidative stress to the increase in the concentration of thioredoxin, and the process going through these plural steps involves individual difference in response, thereby resulting in a drawback that the thioredoxin concentration observed as a result includes an individual fluctuation. It may therefore be difficult, based on such index, to execute a highly precise stress evaluation or to execute a diagnosis of a disease or a progress observation thereof.

Therefore, the present inventors have undertaken an investigation on the relationship between the generation of oxidative stress and the index for evaluation thereof. In the course of such investigation, it is noted that the oxidized form concentration (or oxidized form/reduced form ratio) of thioredoxin responds in fewer steps, in comparison with the thioredoxin concentration, as shown by 1 to 3 below:

1. generation of oxidative stress,

2. erasure of oxidative stress by thioredoxin reduced form and generation of thioredoxin oxidized form, and

3. increase in the thioredoxin oxidized form concentration (or oxidized form/reduced form ratio).

In is further noted that this process does not include steps which are estimated to involve a significant individual difference, such as the detection of oxidative stress by the organism and the induction of thioredoxin. Based on these points, a method of evaluating the oxidative stress utilizing the oxidized form concentration of thioredoxin or a concentration ratio of oxidized form and reduced form as the index has been reached.

In this method, the index is more direct to the oxidative stress as explained above, in comparison with the prior method. For this reason, it is excellent in responsiveness in time from the application of an oxidative stress to the detection thereof. Also, since the thioredoxin oxidized form is generated by an oxidation of thioredoxin and directly reflects the applied oxidative stress, the method has a feature of smaller individual difference in comparison with the measurement of entire amount of thioredoxin in the prior method. As a result, it is made possible to execute a highly precise oxidative stress evaluation or to execute a diagnosis of a disease or a progress observation thereof.

Also the mechanisms of response to various stresses, including oxidative stress, have a mutually common element, and many signal paths are simultaneously activated and execute regulations under close cooperation. Therefore, the method of evaluating the oxidative stress of the present invention may be applicable for evaluating other stresses.

As the index for stress evaluation, the oxidized form concentration of thioredoxin and/or the oxidized form/reduced form ratio may be employed singly or in combination, or preferably employed in combination with another index. Examples of the evaluation method with a combination of indexes include followings:

(1) a method of classifying and evaluating the stress level by quadrants in x-y 2-axis coordinate system;

(2) a method of evaluation by an area of a triangle, formed by x and y coordinates and the original point on a 2-axis coordinate system;

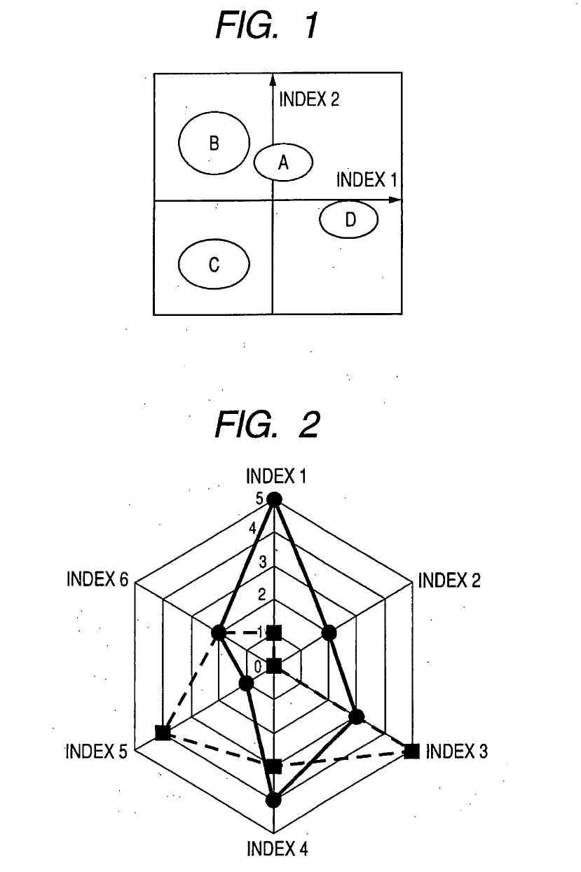

(3) a method of utilizing a coordinate position on a 2-axis coordinate system as shown in FIG. 1;

(4) a method of evaluation by a volume of a tetrahedron, formed by x, y and z coordinates and the original point on a x-y-z 3-axis coordinate system; and

(5) a method of pattern evaluation by a multi-axis pattern shape as shown in FIG. 2.

In case of employing the oxidized form/reduced form ratio of thioredoxin as a single index, a relation between such concentration ratio and the level of stress (stress level) is determined in advance from statistically obtained data. For example, the stress level is divided into 4 ranks of from A to D in the decreasing order of stress level, according to the concentration ratio. The oxidized form/reduced form concentration ratio of thioredoxin in the sample obtained from the subject person of measurement is measured by the aforementioned method, and, based on the obtained measured value, the stress level of the subject person of measurement is classified as one of ranks from A to D. Such classifying process can be automatically executed by processing the measured value of the oxidized form/reduced form concentration ratio by a predetermined computer program. In such case, a CPU of a computer constitutes classifying means. The result of classification may be outputted through a desired medium such as paper or various displays, or may be stored in memory means and may be taken out when necessary. An example of the constituting blocks of such stress level information acquisition apparatus is shown in a block diagram in FIG. 13.

The stress level information acquisition apparatus of the present invention may include, in addition to the classifying means and the output means for the result of classification described above, an information acquisition apparatus for measuring at least one of the oxidized form concentration, the reduced form concentration and the concentration ratio thereof, of thioredoxin. FIG. 14 is a block diagram showing an example of the stress level information acquisition apparatus including the concentration information acquisition apparatus.

The stress level information acquisition apparatus and the stress level judging method of the present invention are applicable advantageously for acquiring information relating to the stress level, useful for diagnosing a disease and for observing a disease progress. Such disease is not particularly restricted as long as it involves a change in the concentration of oxidized form of thioredoxins or in the ratio of oxidized form and reduced form of thioredoxins. Examples of such disease include lung diseases, circulatory diseases, hepatic diseases, digestive tract diseases, kidney diseases, diabetics, aids, tumors and dermatologic diseases.

The stress level information acquisition apparatus and the stress level judging method of the present invention are applicable advantageously, in case of using thioredoxins as a drug, in evaluating or estimating the effect thereof prior to or after the administration thereof.

For example, in the apparatuses shown in FIGS. 13 and 14, a possibility of a disease according to the stress level of the subject person of measurement, judged by the information acquisition apparatus may be displayed on the display device. Such process is made possible by determining a correlation between the stress level and the diseases in advance from statistically collected data and by incorporating, in the CPU, a program which selects possible diseases according to the stress level judged utilizing such data. It is also possible to store the data in time of the subject person of measurement in the memory device, thereby enabling to output the change in the stress level in time of the subject person of measurement, and, based on such change, to display the progress of the related disease in classified manner such as “satisfactory progress” or “no change”. Such classification can also be prepared from a reference prepared from the statistically collected data.

The stress level information acquisition apparatus and the stress level judging method of the present invention are applicable advantageously, in case of using thioredoxins as a drug, in evaluating or estimating the effect thereof prior to or after the administration thereof.

For example, it is also possible to store a pattern of the stress level in which the drug administration is effective, in the memory device of the apparatuses shown in FIGS. 13 and 14, and to compare such pattern and the data from the subject person of measurement, thereby preparing data for assisting the estimation of efficacy of administration in the subject person of measurement.

It is furthermore possible to store, as preserved data, a pattern of change (for example change in time) of the stress level in a case that the drug administration was effective and a case that the drug administration was ineffective in the apparatuses shown in FIGS. 13 and 14, and to execute an evaluation on whether the drug administration was effective or on whether the efficacy can be estimated in advance, by comparing the data from the subject person of measurement subjected to the drug administration with such pattern of change in the stress level, utilizing such preserved data. The pattern of stress level or the pattern of change in the stress level, to be used as reference for such evaluations, may also be prepared from the statistically collected data.

### EXAMPLES

In the following, the present invention will be further clarified by examples, but the present invention is not limited to such examples.

### Example 1

NADPH Enzyme Electrode System

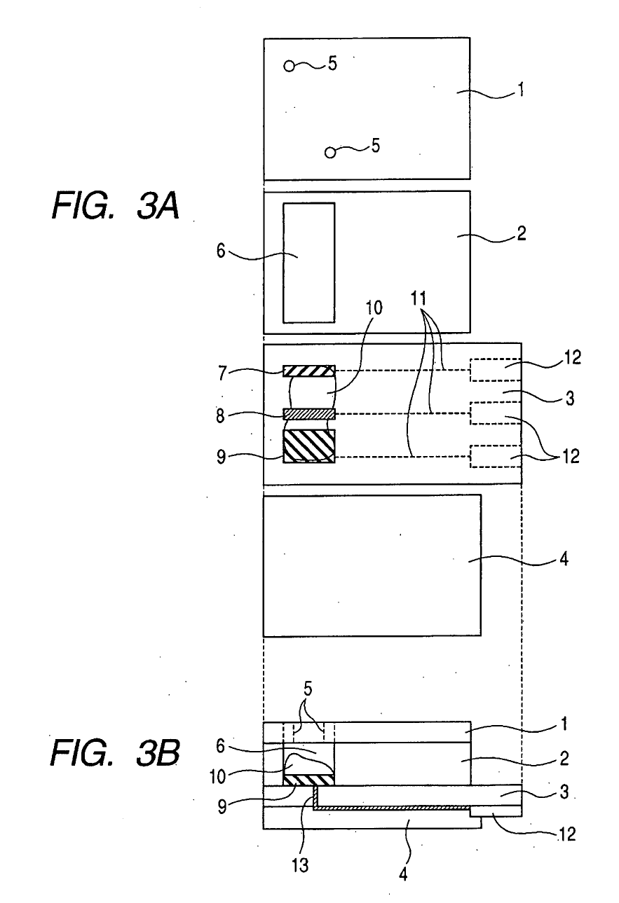

FIGS. 3A and 3B illustrate an example of an apparatus of the present invention for measuring an oxidized form concentration, a reduced form concentration or an oxidized form/reduced form ratio of thioredoxins. FIG. 3A is a plan view showing constituent members developed in an order from above to below. FIG. 3B is a cross-sectional view of the apparatus in a vertical direction. FIGS. 3A and 3B illustrate an example of a basic structure of a sensing part of a thioredoxin oxidized form concentration sensor utilizing an enzyme electrode.

The sensing part in this apparatus is principally constituted of a reaction tank cover 1, a reaction tank wall 2, a substrate 3, and an insulating layer 4. The reaction tank cover 1 is formed for example of a polyethylene terephthalate (PET) resin bearing a pressure-sensitive adhesive material, and is provided with apertures 5 constituting a sample inlet and an air outlet, respectively positioned in diagonal positions of a reaction tank 6. The reaction tank wall 2 is formed for example of polydimethylsiloxane (PDMS) and is designed as to hold the sample of a predetermined amount (for example 500 μL) on electrodes 7, 8, 9 of the substrate 3. The substrate 3 is formed by a plate member for example of polyimide, and the substrate 3 is provided thereon with a working electrode 8, a counter electrode 9 and a reference electrode 7. Each electrode is connected through a through hole 13 to a rear surface of the substrate 3, and is connected through a lead 11 to a current collecting pad 12. The through holes 13 and the leads 11 are formed in advance by plating and photolithographic process on a copper-clad polyimide substrate.

The working electrode 8 is formed by a glassy carbon electrode subjected to a polyaminoaniline treatment. A thin piece is cut out from a glassy carbon rod, and is temporarily adhered with a conductive paste onto a temporary substrate (not shown). A lead wire is connected to the rear surface, and a potential is applied by a potentiostat to the cylindrical thin piece of glassy carbon, to constitute the working electrode 8, utilizing a reference electrode and a counter electrode (not shown) for polyaminoaniline treatment in a 1.0 M aqueous solution of sulfuric acid containing 0.01 M of 2-nitroaniline. After repeating a cycle of applying 1.5 V for 10 seconds and −0.5 V for 50 seconds over one hour, a potential of −0.5 V is applied for 10 minutes, followed by a rinsing with water, whereby a working electrode is obtained. After the rinsing with water, the working electrode is detached from the temporary substrate and is adhered by a conductive paste onto the sensor substrate 3. The counter electrode 9 is formed by sputtering a Ti/Pt film on the substrate 3. An example of the film thickness is 100 nm of Ti and 200 nm of Pt. The reference electrode 7 is prepared by forming a Ti/Pt film in the same manner as the counter electrode 9, and by sputtering an Ag layer, followed by a chlorination treatment. An example of the thickness of Ag layer is 500 nm. The thickness of the Ag layer has to be optimized depending on the environment and the time of use. The chlorination treatment for the Ag layer is executed by an immersion in a 50 mM aqueous solution of FeCl₃ for 10 minutes. On each electrode, a reagent layer of a predetermined composition and a predetermined amount is coated. The reagent layer is prepared in advance by mixing an enzyme of a predetermined amount, with an enzyme carrier and a mediator molecules if necessary, so as to easily dissolve out into an aqueous solution. The method of preparation of the reagent layer will be explained below.

An aqueous solution is prepared by mixing 50 nmol (corresponding to viologen molecules) of a viologen derivative immobilized on alginic acid, 1 μmol of NADPH, 0.1 units of ferredoxin NADP+reductase and 1 unit of thioredoxin reductase. The dry layer is formed by dropping the aqueous solution onto the working electrode 8, followed by drying.

Method of synthesizing viologen derivative, immobilized in the alginic acid layer, will be explained below.

4,4′-bipyridine is added with methyl iodide and 1,4-dibromobutane of equimolar amounts and is reacted in an autoclave at 110° C. for 6 hours. The raw materials are removed by distillation under a reduced pressure from the obtained product, and a water-soluble component by a silica gel column to obtain desired bromide/iodide salt of 1-methyl-1-1′-bromobutyl-4,4′-bipyridine (BrBuV). 28.7 mg of commercial sodium alginate (molecular weight 20,000) and 0.15 mmol of 1-ethyl-3-(3-dimethylaminopropyl)carbodiimide hydrochlorate (EDC) are dissolved in 20 mL of water. After agitation for 40 minutes, 0.75 mmol of commercial polyethylene oxide diamine are added and the mixture is further agitated for 1 hour. The solution is dialyzed with water for 24 hours, and, after addition of 0.3 mmol of BrBuV, further dialyzed with water for 12 hours at to 5° C. to obtain a viologen derivative immobilized on alginic acid.

Prior to measurement, an oxidized form and a reduced form of thioredoxin are prepared. The oxidized form of thioredoxin is obtained by adding hydrogen peroxide of a sufficient amount to a phosphate buffer containing commercial thioredoxin and peroxiredoxin of a sufficient amount thereby oxidizing thioredoxin, followed by a separation by gel permeation chromatography. On the other hand, the reduced form of thioredoxin is obtained by adding NADPH of a sufficient amount to a phosphate buffer containing thioredoxin reductase of a sufficient amount thereby reducing thioredoxin, followed by a separation by gel permeation chromatography.

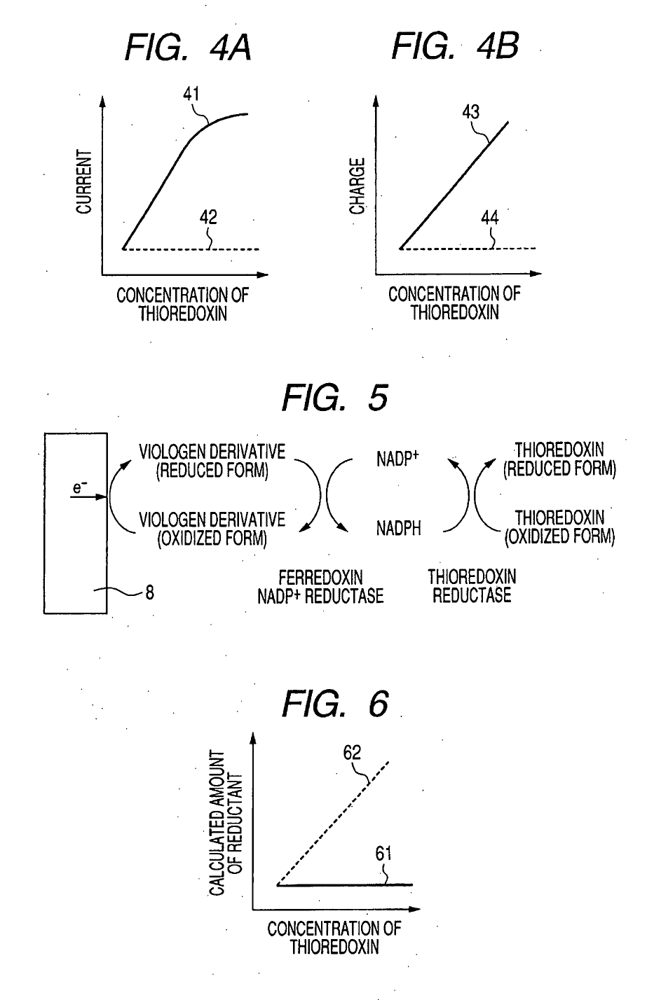

The measuring unit is connected to a potentiostat, and a solution containing thioredoxin oxidized form in a 50 mM phosphate buffer of pH 7.0 and a solution containing thioredoxin reduced form in a 50 mM phosphate buffer of pH 7.0 are respectively prepared. The prepared solution, after a temperature adjustment to 37° C. and a bubbling with nitrogen, is poured through the inlet to the measuring part, and a potential of −0.9 V with respect to the reference electrode is applied to the working electrode. By plotting the stationary current or the cumulative charge amount observed on the ordinate and the concentration of the added oxidized form or reduced form of thioredoxin on the abscissa, the observed charge amount shows a behavior as shown in FIGS. 4A and 4B. In the figures, the curves 41 and 43 illustrate the oxidized forms and the curves 42 and 44 illustrate the reduced forms. More specifically, in the sample prepared by adding thioredoxin oxidized form to the buffer, the stationary current or the charge amount increases with an increase in the thioredoxin concentration. On the other hand, in the sample prepared by adding thioredoxin reduced form to the buffer, the stationary current or the charge amount does not increase with an increase in the thioredoxin concentration. In the case of oxidized form, a reaction as shown in FIG. 5 proceeds in which the thioredoxin oxidized form receives an electron from NADPH by the catalytic action of thioredoxin reductase, thereby becoming a reduced form. The amount of such reaction is proportional to the amount of thioredoxin oxidized form in the solution, namely the amount of thioredoxin that can be reduced in the solution. On the other hand, in the case of reduced form, thioredoxin already in a reduced form state cannot receive an electron from NADPH. Also in case of measuring the reduced form amount (R/mol) of thioredoxin with this measuring unit, a predetermined amount of peroxiredoxin and hydrogen peroxide of a molar amount larger than the anticipated amount of thioredoxin reduced form are added to the sample solution in advance, as a pre-treatment, thereby converting the thioredoxin reduced form in the solution into oxidized form. Thereafter, catalase is added to the system to decompose excessive H₂O₂. Then the charge amount (X/C) is measured in the same manner as in the measurement of thioredoxin oxidized form amount. Thus the amount of reduced form in the sample can be determined, utilizing the thioredoxin oxidized form amount (O/mol) and X in the solution, according to an equation (R)=(X/2F)−(O) (F being Faraday constant). In this case, a plotting of the calculated reduced form amount on the ordinate and the thioredoxin concentration of oxidized form or reduced form solution on the abscissa shows a behavior as shown in FIG. 6. In the figure, the curve 61 illustrates the oxidized form and the curve 62 illustrates the reduced form.

### Example 2

NADPH Colorimetry System

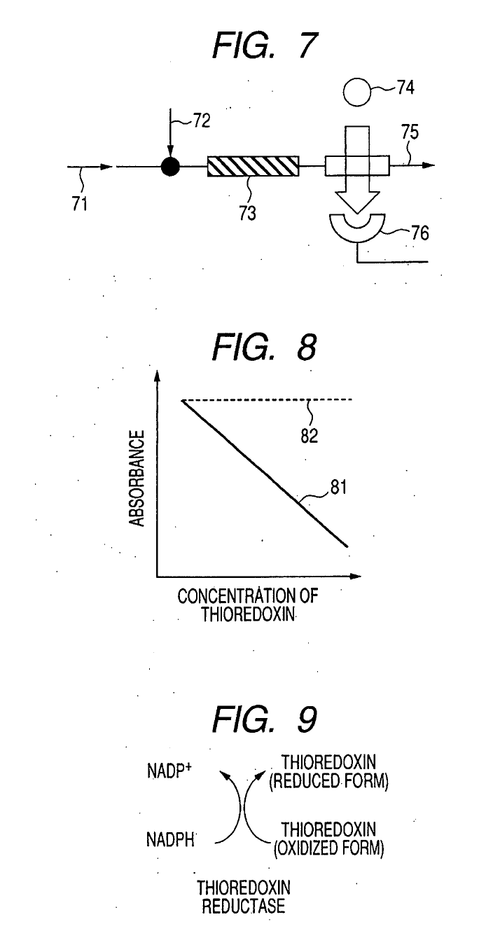

FIG. 7 illustrates an apparatus of the present invention for measuring a concentration of oxidized form or reduced form of thioredoxin or a concentration ratio of oxidized form and reduced form, and more specifically explains a basic step of a thioredoxin oxidized form/reduced form concentration sensor utilizing a colorimetric method. The sensor includes a cell constituting a reaction space, a light source for measuring an optical change based on a reaction taking place in the cell, and detection means including a photosensor element. The measuring process utilizing such sensor is principally constituted of an optical cell introducing step, a sample introducing step, a reaction step, a detection step and a discharge step. In the optical cell introducing step, an optical cell containing a reaction reagent is fixed on a holder. Examples of the reaction reagent include a phosphate buffer containing thioredoxin reductase and NADPH. Concentration and amount are for example 0.5 mL of a 0.1 M phosphate buffer of pH 7.0, containing 0.1 units of thioredoxin reduction enzyme and 50 nmol of NADPH. In the sample introducing step, a sample is introduced into the optical cell. The amount of the introduced sample is for example 0.5 mL. In the reaction step, the solution is agitated and the cell is maintained at a temperature suitable for an enzyme reaction (for example 25° C.) The reaction time may be a time sufficient for converting all the oxidized form of thioredoxin into the reduced form (for example 20 minutes), or the absorbance may be observed during the change, under exact measurement of time from the sample introduction. In the detection step, the optical cell is transmitted by a light, spectrally separated by a grating or the like, from a monochromatic light source, or a multi-colored light source and the transmitted light is detected by the photosensor element. In the discharge step, the optical cell after the measurement is discharged.

As the reaction reagent, 0.5 mL of a 0.1 M phosphate buffer of pH 7.0 containing 0.1 units of thioredoxin and 50 nmol of NADPH are used. As the sample, 0.5 mL each of a 0.1 M phosphate buffer of pH 7.0 containing the thioredoxin oxidized form or reduced form in an amount of from 0 to 10 nmol are used. On each sample, an absorbance observed in the detection part after a reaction time of 20 minutes at 25° C. On the other hand, as a comparative reference sample, 0.5 mL of a phosphate buffer of pH 7.0 not containing thioredoxin are used, and an absorbance observed in the detection part after a reaction time of 20 minutes at 25° C. A plotting of the value of (comparative reference)-(sample) of the observed absorbances on the ordinate and the concentration of added thioredoxin on the abscissa shows a behavior as shown in FIG. 8. In the figure, the curve 81 illustrates the oxidized form and the curve 82 illustrates the reduced form. NADPH has an absorption of an absorption coefficient of 6220 M⁻¹cm⁻¹ at 340 nm, but its oxidized form NADP⁺ does not have the absorption. Thus, in the sample prepared by adding thioredoxin oxidized form to the buffer, because of the change from NADPH to NADP⁺, the absorbance at 340 nm decreases with respect to the comparative reference with an increase in the thioredoxin concentration. On the other hand, in the sample prepared by adding thioredoxin reduced form to the buffer, NADPH concentration does not change by an increase in the thioredoxin concentration, whereby the absorbance does not change. In the solution in which the thioredoxin oxidized form is added, a reaction as shown in FIG. 9 proceeds, in which the thioredoxin oxidized form receives an electron from NADPH through the catalytic action of thioredoxin reductase, thereby becoming reduced form. The amount of such reaction is proportional to the amount of thioredoxin oxidized form in the solution, namely to the thioredoxin that can be reduced in the solution. On the other hand, in the case of reduced form, thioredoxin, being already in the reduced form state, cannot receive an electron from NADPH.

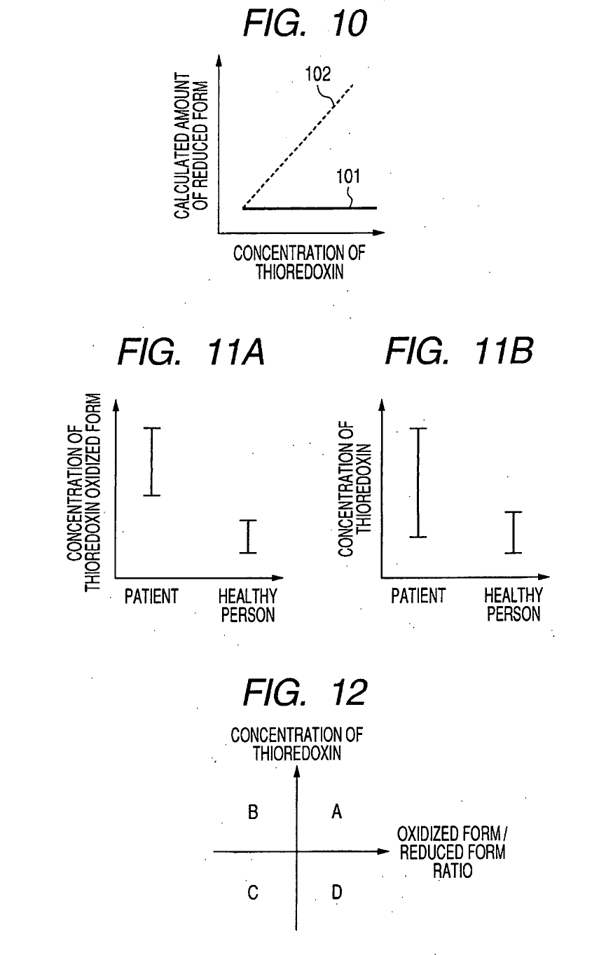

In case of measuring the reduced form amount (R/mol) of thioredoxin with this measuring unit, a predetermined amount of peroxiredoxin and hydrogen peroxide are added to the sample solution in advance, as a pre-treatment, thereby converting the thioredoxin reduced form in the solution into oxidized form. Thereafter, the absorbance is measured in the same manner as in the measurement of thioredoxin oxidized form amount, and a difference (Y) from the comparative reference is calculated. Thus the amount of reduced form can be determined, utilizing the amount of added hydrogen peroxide (H/mol), the thioredoxin oxidized form amount (0/mol) in the solution and NADPH amount (Y′/mol) corresponding to Y, according to an equation (R)=(H)+(O)−(Y′). In this case, a plotting of the calculated reduced form amount on the ordinate and the thioredoxin concentration of oxidized form or reduced form solution on the abscissa shows a behavior as shown in FIG. 10. In the figure, the curve 101 illustrates the oxidized form and the curve 102 illustrates the reduced form.

### Example 3

Pre-Treatment

Blood sample is collected from the arm of a hepatitis C patent or a healthy person and is centrifuged for 10 minutes at 3000 rpm to obtain a serum. Concentrations of oxidized form and reduced form in the serum are measured with the electrode of Example 1. On the other hand, a thioredoxin concentration of the same serum is measured by a commercial sandwich ELISA method (kit available from Redox Bioscience Inc.). The results of measurement shows a behavior as shown in FIG. 11. More specifically, the case of utilizing the thioredoxin oxidized form concentration as the index shows a smaller individual difference and an improved response to the oxidative stress, in comparison with the case of utilizing the total thioredoxin concentration as the index. This is estimated because the relationship between the measured total thioredoxin concentration and the oxidative stress is an indirect relation involving plural steps such as 1. generation of oxidative stress, 2. detection of oxidative stress by the organism, 3. induction of thioredoxin, and 4. an increase in the thioredoxin concentration, while the thioredoxin oxidized form concentration shows a direct increase by oxidation of thioredoxin, thereby directly reflecting the applied oxidative stress. Therefore there is enabled an evaluation of oxidative stress, with a smaller individual difference and a better response, in comparison with the case of measuring the total thioredoxin amount.

### Example 4

Stress Evaluation

FIG. 12 shows, as an example of oxidative stress evaluation, a plotting of the total thioredoxin concentration on the ordinate and the oxidized form/reduced form ratio of thioredoxin on the abscissa. A person belonging to an area indicated by a symbol A is considered to have a possibility of being subjected to a strong oxidative stress, and when it is derived from a disease, an improvement in symptoms is considered possible by an administration of thioredoxin as a drug. Also a person belonging to an area indicated by a symbol B is considered to have a possibility, despite of the high thioredoxin concentration, of not subjected to a strong oxidative stress or having once been subjected to a strong oxidative stress, which is now weaker. Also in the case that the person belonging to the area indicated by the symbol B has a disease which generates an oxidative stress, the possibility of an improvement in symptoms is considered not high by an administration of thioredoxin as a drug. This is because, in a situation where the body is already rich in thioredoxin reduced form, the possibility of reducing oxidative stress is not so high by an additional administration of thioredoxin from the exterior. Then a person belonging to an area indicated by a symbol C is considered to have a high possibility not subjected to a strong oxidative stress, based on either index. Finally, a person belonging to an area indicated by a symbol D has a high possibility not subjected to a strong oxidative stress, based on the total thioredoxin concentration only. However, there is considered a possibility of being recently subjected to a strong oxidative stress, namely a possibility in a transient period after thioredoxin induction but before the increase of the total thioredoxin concentration, or a possibility of a body constitution of a low thioredoxin inducing ability. In the case that the person belonging to the area indicated by the symbol D has a disease which generates an oxidative stress, the possibility of an improvement in symptoms is considered high by an administration of thioredoxin as a drug.

Thus, an oxidized form/reduced form ratio of thioredoxin is introduced as an additional index, in comparison with the case of utilizing the total thioredoxin concentration as the index. As a result, it is rendered possible to obtain new and useful data such as an influence of a current oxidative stress to the organism, a timing of application of oxidative stress, and an efficacy of thioredoxin used as a drug.

### Example 5

Reduced Form Measuring System with Enzyme Electrode

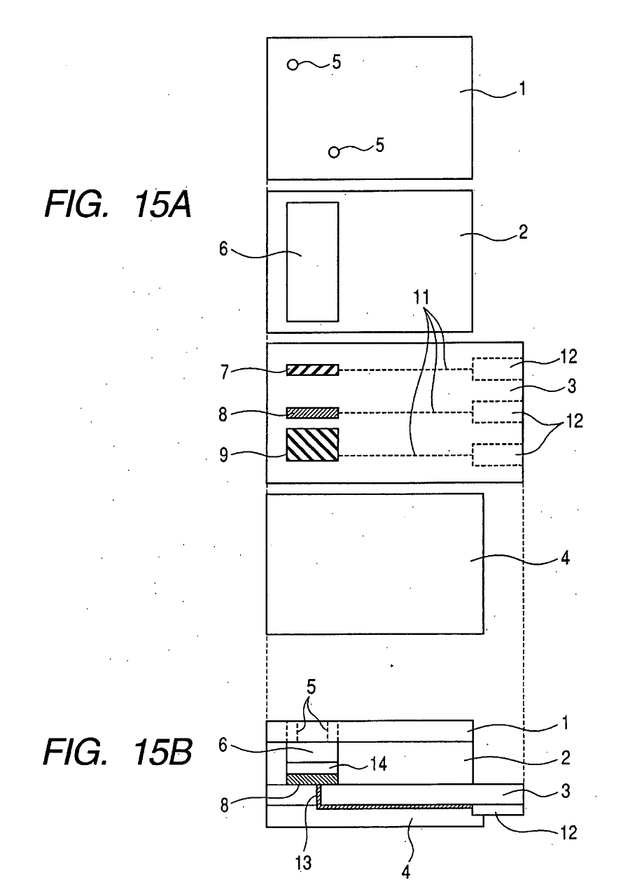

FIGS. 15A and 15B are views showing an example of an apparatus of the present invention for measuring an oxidized form concentration, a reduced form concentration or an oxidized form/reduced form concentration ratio of thioredoxins. The basic structure of measurement electrode part is same as that shown in FIGS. 3A and 3B, except that the working electrode 8 is different in the preparation method, the reagent layer 10 is absent and an enzyme layer 14 is present. The method of preparation will be explained below, with emphasis on these differences.

The working electrode 8 is formed for example of a carbon electrode, a polymer containing an osmium complex and an enzyme immobilized thereto. The preparation method thereof will be explained below.

A commercial carbon paste is coated by a screen printing onto the substrate 3, and is hydrophilized by a UV-O₃ treatment. A mixture of a polymer containing an osmium complex, an enzyme and an aqueous solution of a crosslinking agent is dropped (for example with an amount of 50 μLcm⁻²) thereon and dried to obtain a working electrode. The liquid to be dropped is constituted for example of a compound 1 of following structure, horseradish peroxidase and polyethylene glycol diglycidyl ether, for example with respective concentrations of 5 mgmL⁻¹, 1 mgmL⁻¹ and 0.2 mgmL⁻¹.

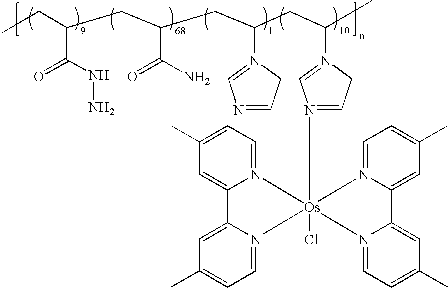

Preparation method of the compound 1 will be explained below.

In a 100-mL eggplant-shaped flask equipped with a reflux condenser, 20 mL of ethylene glycol and 0.08 g of (NH₄)₂[OsCl₆], and 0.38 g of 4,4′-dimethyl-2,2′-bipyridine were charged. Then, under agitation by a stirrer and under a nitrogen gas flow, a microwave irradiation of 300 W was conducted for 20 minutes by a microwave synthesizer (Milestone microsynth). After the solution was cooled to the room temperature, 25 mL of water containing 0.4 g of Na₂S₂O₄ were added. After agitation for 1 hour at the room temperature, the resulting black-purple precipitate was separated by filtration and rinsed with water to remove excessive salt. Then it was rinsed with diethyl ether to remove the unreacted ligand, and was dried by heating to 60° C. under a reduced pressure to obtain Os(4,4′-dimethyl-2,2′-bipyridine)₂Cl₂.

In a 100-mL three-necked flask equipped with a thermometer and a reflux condenser, 15 mL of water, 2.63 g of acrylamide, 0.403 mL of 1-vinylimidazole, and 0.069 mL of N,N,N′,N′-tetramethylethylenediamine were charged. Under a nitrogen gas flow, 0.06 g of ammonium persulfate were further added. The reactor was heated at 40° C. on a water bath for 30 minutes, and was then air cooled. The resulting viscous liquid was dropwise added to 500 mL of methanol under strong agitation to cause sedimentation, and the sediment was collected by centrifuging and dissolved by adding water of a minimum amount necessary for dissolving the precipitate, and the aqueous solution was dropwise added to 500 mL of methanol under strong agitation to cause sedimentation. The sediment was recovered again by centrifuging, and dried by heating at 60° C. under a reduced pressure thereby obtaining a polyacrylamide-polyvinylimidazole 7.49/1 copolymer. The molecular generation and the unit ratio were confirmed by ¹HNMR (D₂O) measurement.

In a 100-mL eggplant-shaped flask with a reflux condenser, 25 mL of ethylene glycol, 17.5 mL of ethanol, 0.19 g of Os(4,4′-dimethyl-2,2′-bipyridine)₂Cl₂ prepared above, and 0.22 g of polyacrylamide-polyvinylimidazole copolymer were charged. Then, under agitation by a stirrer and under a nitrogen gas flow, a microwave irradiation of 400 W was conducted for 2 hours by a microwave synthesizer. After the solution was cooled to the room temperature, a solution obtained by adding 20 mL of ethanol was dropwise added to 500 mL of diethyl ether under strong agitation, and 20 mL of ethanol were further added to the resulting viscous precipitate. It was again dropwise added to 500 mL of diethyl ether solution under strong agitation, and the resulting viscous precipitate was dried by heating at 60° C. under a reduced pressure to obtain a desired complex polymer of the formula (1).

The enzyme layer 14 is formed for example of a polyvinylidene fluoride film carrying peroxiredoxin. A carrying amount is for example 250 unit·cm². It may be prepared by dropwise adding an aqueous enzyme solution onto the film, followed by a drying. The film is so disposed as to cover the working electrode 8.

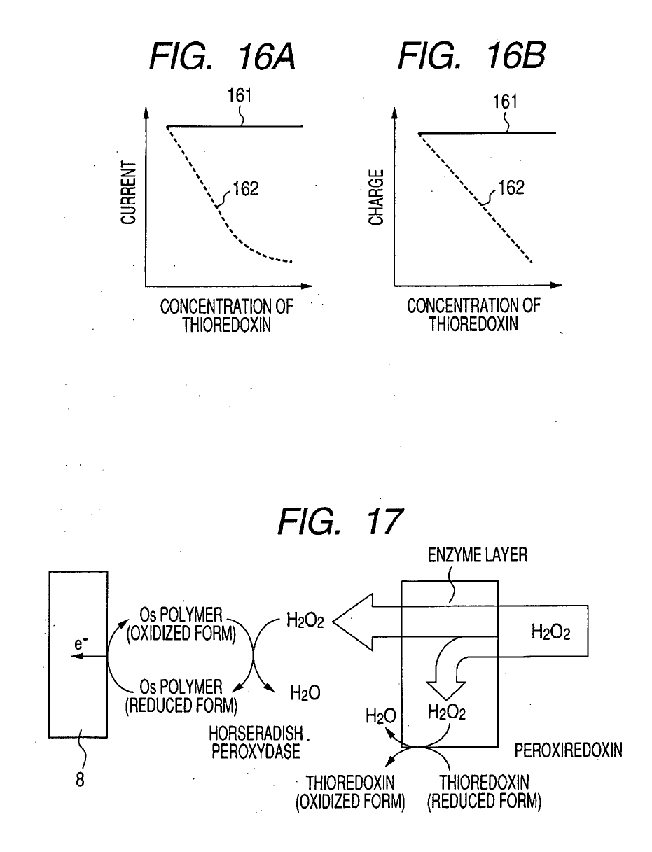

The measuring unit is connected to a potentiostat, and a solution containing thioredoxin oxidized form in a 50 mM phosphate buffer of pH 7.0 and a solution containing thioredoxin reduced form in a 50 mM phosphate buffer of pH 7.0 are respectively prepared. The prepared solution, after a temperature adjustment to 37° C. and a bubbling with nitrogen, is poured through the inlet to the measuring unit, and an aqueous solution of hydrogen peroxide in a molar amount of one to several times with respect to the anticipated molar amount of thioredoxin. Then a potential of +0.2 V with respect to the reference electrode is applied to the working electrode. By plotting the stationary current or the cumulative charge amount observed on the ordinate and the concentration of the added oxidized form or reduced form of thioredoxin on the abscissa, the observed charge amount shows a behavior as shown in FIGS. 16A and 16B. In the figures, the curves 161 illustrate the oxidized forms and the curves 162 illustrate the reduced forms. More specifically, in the sample prepared by adding thioredoxin reduced form to the buffer, the stationary current or the charge amount decreases with an increase in the thioredoxin concentration. On the other hand, in the sample prepared by adding thioredoxin oxidized form to the buffer, the stationary current or the charge amount does not decrease with an increase in the thioredoxin concentration. These phenomena can be explained with reference to FIG. 17. In the absence of thioredoxin reduced form, the oxidation reaction of H₂O₂ in the solution can be observed as a current or a charge on the electrode, by a catalytic reaction of horseradish peroxidase and an electron transferring function of osmium complex. In the presence of thioredoxin reduced form in the system, a reaction of consuming H₂O₂ in the solution by thioredoxin reduced form proceeds in the enzyme layer, by the catalytic function of peroxiredoxin. Therefore, in the presence of thioredoxin reduced form, the current or charge resulting from the oxidation reaction of H₂O₂ decreases, and the amount of such decrease is proportional to the amount of thioredoxin reduced form in the solution, namely the amount of thioredoxin that can be oxidized in the solution. On the other hand, in the case of oxidized form, thioredoxin already in an oxidized form state can no longer be oxidized even in the presence of horseradish peroxidase, whereby the stationary current or the charge amount does not decrease even by increasing the thioredoxin oxidized form concentration.

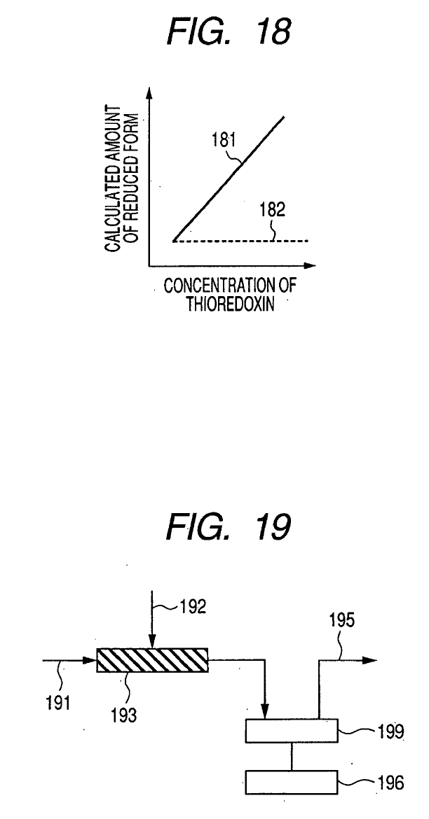

Also in case of measuring the oxidized form amount (O/mol) of thioredoxin with this measuring unit, a predetermined amount of thioredoxin reductase and NADPH of a molar amount larger than the anticipated amount of thioredoxin oxidized form are added to the sample solution in advance, as a pre-treatment, thereby converting the thioredoxin oxidized form in the solution into reduced form. Thereafter, NADPH oxidase and oxygen are introduced into the system to convert excessive NADPH into NADP⁺. Then the charge amount (X/C) is measured in the same manner as in the measurement of thioredoxin reduced form amount. Thus the amount of oxidized form in the sample can be determined, utilizing the thioredoxin reduced form amount (R/mol) and X in the solution, according to an equation (O)=(X/2F)−(R) (F being Faraday constant). In this case, a plotting of the calculated reduced form amount on the ordinate and the thioredoxin concentration of oxidized form or reduced form solution on the abscissa shows a behavior as shown in FIG. 18. In the figure, the curve 181 illustrates the oxidized form and the curve 182 illustrates the reduced form.

### Example 6

NADPH Modification Electrode System

FIG. 19 illustrates an apparatus of the present invention for measuring a concentration of oxidized form or reduced form of thioredoxin or a concentration ratio of oxidized form and reduced form, and more specifically explains a basic step of a thioredoxin oxidized form/reduced form concentration sensor utilizing a calorimetric method. The sensor includes a cell constituting a reaction space, and a detection means including an electrochemical measuring cell for measuring an electrochemical change based on a reaction taking place in the cell. The measuring process utilizing such sensor is principally constituted of a sample introducing step, a reagent introducing step, a reaction step, a reaction solution introducing step, a detection step and a discharge step.

In the sample introducing step 191, a specific amount of a sample to be measured is introduced into a reactor 193.

In the reagent introducing step 192, a reagent used for the reaction is introduced into the reactor. Examples of the reaction reagent include a phosphate buffer containing thioredoxin reductase and NADPH.

In the reaction step, the solution is agitated and the cell is maintained at a temperature suitable for an enzyme reaction to react the sample with the added reagent. The reaction time may be a time sufficient for converting all the oxidized form of thioredoxin into the reduced form (for example 20 minutes), or the signal may be observed during the change in a succeeding detecting step, under exact measurement of time from the sample introduction.

In the reaction solution introducing step, in case that the electrochemical cell 199 does not serve as the reaction cell, the solution after the reaction is transferred to the electrochemical cell. In the detection step, a potential is applied to a gold electrode by a potentiostat 196 to detect and record a responsive current and charge.

Then the solution after the measurement is discharged in the discharge step.

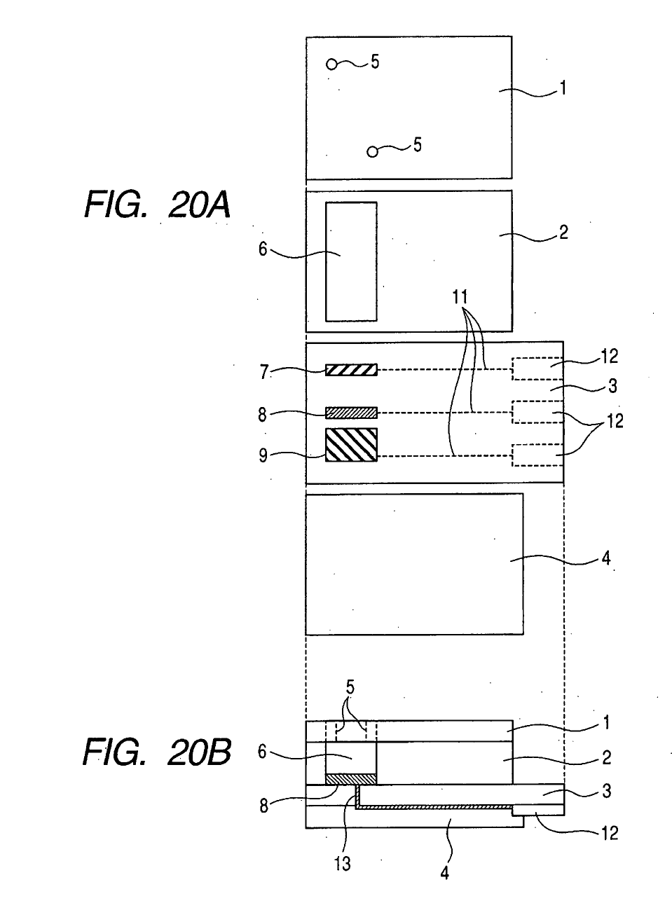

FIGS. 20A and 20B illustrate an example of an apparatus of the present invention for measuring an oxidized form concentration, a reduced form concentration or an oxidized form/reduced form ratio of thioredoxins. The fundamental structure of the measurement electrode part is the same as illustrated by FIGS. 3A and 3B except that the preparing method of reaction electrode 8 and that there is no reagent layer 10. The preparation method is explained below, mainly directing the viewpoint toward the differences. The reaction electrode 8 is comprised of, for example, a gold electrode and a modified molecule on the gold electrode, e.g. pyrroloquinoline quinone (PQQ). The process for preparing it is explained below.

A gold layer is formed on a substrate 3 having a titanium as a base layer by vapor deposition, sputtering and so forth. The gold electrode is purified by UV-O₃ treatment, followed by making a cystameine aqueous solution drop thereon, washing the resultant with water, making a buffer solution containing a coupling agent N-(3-Dimethylaminopropyl)-N′-ethylcarbodiimide hydrochloride (EDC) for PQQ drop thereon, and washing the resultant with the buffer solution.

As the reagent, 0.5 mL of a 0.1 M phosphate buffer of pH 7.0 containing 0.1 units of thioredoxin reducing enzyme and 50 nmol of NADPH are used.

As the sample, 0.5 mL each of a 0.1 M phosphate buffer of pH 7.0 containing the thioredoxin oxidized form or reduced form in an amount of from 0 to 10 nmol are used. On each sample, a current or charge observed in the detection part after a reaction time of 20 minutes at 25° C. is measured. On the other hand, as a comparative reference sample, 0.5 mL of a phosphate buffer of pH 7.0 not containing thioredoxin are used, and a current or charge observed in the detection part after a reaction time of 20 minutes at 25° C. is measured.

The electrode unit is connected to a potentiostat. A reaction solution, after a temperature adjustment to 37° C. and a bubbling with nitrogen, is poured through the inlet to the measuring part, and a potential of 0.2 V with respect to the reference electrode is applied to the working electrode.

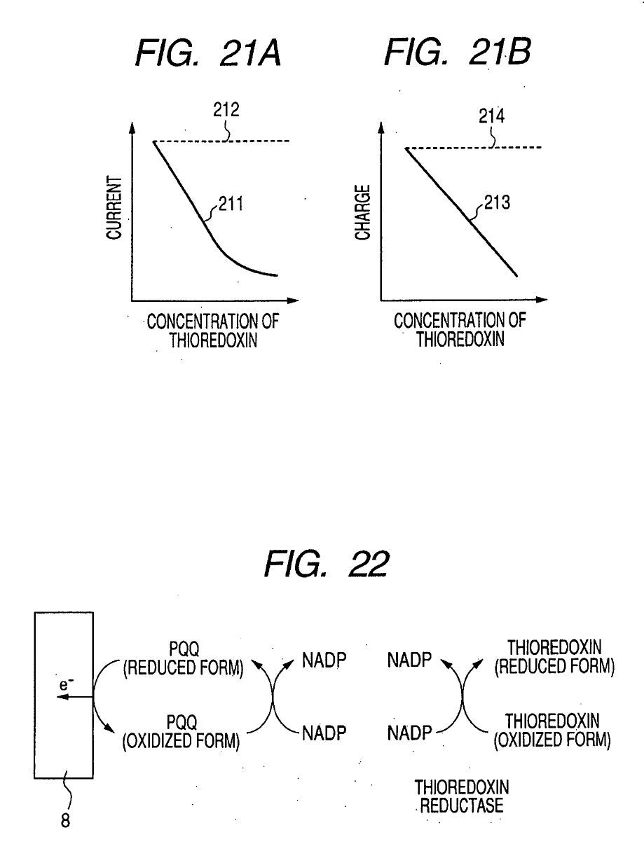

By plotting the stationary current or the cumulative charge amount observed on the ordinate and the concentration of the added oxidized form or reduced form of thioredoxin on the abscissa, the observed charge amount shows a behavior as shown in FIGS. 21A and 21B. In the figures, the curves 211 and 213 illustrate the oxidized forms and the curves 212 and 214 illustrate the reduced forms.

In the sample prepared by adding thioredoxin oxidized form to the buffer, the stationary current or the charge amount increases with an increase in the thioredoxin concentration. On the other hand, in the sample prepared by adding thioredoxin reduced form to the buffer, the stationary current or the charge amount does not increase with an increase in the thioredoxin concentration. The phenomenon is more explained below, using FIG. 22.

On the electrode, a current caused by the oxidation reaction of NADPH through PQQ is observed. The amount of the current or the charge increase or decrease in proportion to the concentration of NADPH in the solution.

While the thioredoxin in oxidized form is reduced by NADPH with the aid of catalysis of thioredoxin reductase and as a result the concentration of NADPH in the solution is decreased, the thioredoxin in reduced form is not able to be reduced by NADPH any longer so that the concentration of NADPH in the solution is not decreased.

It is utilized for quantifying the amount (R/mol) of thioredoxin in reduced form by this sensor to change previously the thioredoxin in reduced form in the solution into the oxidized form as a pretreatment.

The present invention allows to provide an apparatus capable of measuring an oxidized form concentration, a reduced form concentration or an oxidized form/reduced form concentration ratio of thioredoxins, and such apparatus can be utilized advantageously as an apparatus for stress evaluation, utilizing these parameters as indexes.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-034350, filed Feb. 10, 2006, which is hereby incorporated by reference herein in its entirety.

What is claimed is:

1. An information acquisition apparatus for acquiring information on a concentration of thioredoxins, wherein a concentration of an oxidized form and/or reduced form of thioredoxins in a sample is measured, utilizing an redox reaction of the thioredoxins and distinguishing the oxidized form and the reduced form.

2. The information acquisition apparatus according to claim 1, which comprises:
a reaction space for reacting the sample and an enzyme for the redox reaction of thioredoxins, and
a detection means for calculating at least one selected from the group consisting of the oxidized form concentration, the reduced form concentration and a ratio of the concentrations, based on the reaction in the reaction space.

3. The information acquisition apparatus according to claim 1, wherein the enzyme is a thioredoxin reductase.

4. The information acquisition apparatus according to claim 1, wherein the enzyme is an immobilized enzyme immobilized on a carrier, and disposed in the reaction space.

5. The information acquisition apparatus according to claim 1, wherein the detection means is adapted to detect an optical change based on the reaction and to calculate at least one selected from the group consisting of the oxidized form concentration, the reduced form concentration and a ratio of the concentrations.

6. The information acquisition apparatus according to claim 1, wherein the detection means is adapted to detect an electrochemical change based on the reaction and to calculate at least one selected from the group consisting of the oxidized form concentration, the reduced form concentration and a ratio of the concentrations.

7. The information acquisition apparatus according to claim 6, wherein the detection means comprises an enzyme electrode.

8. The information acquisition apparatus according to claim 6, wherein the electrochemical change to be detected is at least one selected from the group consisting of current, charge amount, potential, voltage and impedance.

9. A stress sensor comprising an information acquisition apparatus according to claim 1, and a stress level judging unit.

10. A stress level information acquisition apparatus for acquiring information on a stress level in a subject person of measurement, comprising a stress level judging means which measures a concentration of an oxidized form and/or reduced form of thioredoxins in a sample derived from a subject person of measurement, utilizing an redox reaction of the thioredoxins and distinguishing the oxidized form and the reduced form, and judges a stress level of the subject person of measurement based on the first information selected from the group consisting of the oxidized form concentration, the reduced form concentration and a ratio of the concentrations and on the second information concerning a relation between the first information and a stress level.

11. A stress level information acquisition apparatus according to claim 10, further comprising judgment result output means which outputs a result of stress level judged by the stress level judging means, and display means which displays the outputted judgment result.

12. A stress level information acquisition apparatus according to claim 10, further comprising memory means which stores the output from the judgment result output means, wherein a change in time in the stress level of the subject person of measurement is outputted from the judgment result output means based on the judgment results accumulated in the memory means.

13. A stress level information acquisition apparatus according to claim 10, wherein at least either of an oxidized form concentration of thioredoxins and a concentration ratio of the oxidized form and the reduced form in the sample of the subject person of measurement is utilized for judging the stress level.

14. A stress level judging method for judging a stress level in a subject person of measurement, comprising a stress level judging step of judging a stress level, based, utilizing an redox reaction of thioredoxins in a sample derived from the subject person of measurement, on at least either of an oxidized form concentration and a reduced form concentration of the thioredoxins and a preset standard.

15. The stress level judging method according to claim 14, wherein at least either of an oxidized form concentration of thioredoxins and a concentration ratio of the oxidized form and the reduced form in the sample of the subject person of measurement is utilized for judging the stress level.

16. An enzyme electrode comprising a conductive member and an enzyme, wherein the enzyme catalyzes an redox reaction of thioredoxins.

17. An enzyme electrode according to claim 16, wherein the enzyme is a thioredoxin reductase.

18. A thioredoxin concentration measuring method for measuring a concentration of thioredoxins in a sample, which comprises, utilizing an redox reaction of thioredoxins in the sample, to measure at least either of an oxidized form concentration and a reduced form concentration of the thioredoxins in distinguished manner.

19. The measuring method according to claim 18, wherein the enzyme is a thioredoxin reductase.


SEQUENCE LISTING (1 sequence)

SEQ ID NO: 1 - protein, 4 aa, Artificial
  sequence comprised in thioredoxin
CXXC